(19) United States
(12) Patent Application Publication
(10) Pub. No.: US 2018/0200513 A1
(43) Pub. Date: Jul. 19, 2018

(54) MODULATION OF THE SUPERIOR CERVICAL GANGLION FOR THE TREATMENT OF INSOMNIA

(71) Applicant: Boston Scientific Neuromodulation Corporation, Valencia, CA (US)

(72) Inventors: Kyle Harish Srivastava, Saint Paul, MN (US); Bryan Allen Clark, Forest Lake, MN (US); William Conrad Stoffregen, Lake Elmo, MN (US)

(21) Appl. No.: 15/873,449

(22) Filed: Jan. 17, 2018

Related U.S. Application Data
(60) Provisional application No. 62/447,680, filed on Jan. 18, 2017.

(51) Int. Cl. A61N 1/36; A61B 5/00; A61N 1/375

(52) U.S. Cl. CPC A61N 1/3603; A61B 5/414; A61B 5/0031; A61N 1/36125; A61N 1/3606; A61N 1/37514

(57) ABSTRACT

This document discusses, among other things, systems and methods to electrically modulate a patient's pineal gland to control release of melatonin from the pineal gland and regulate melatonin levels of the patient, the electrically modulating the patient's pineal gland including delivering a neuromodulation waveform using at least one electrode to modulate a superior cervical ganglion or ganglia.

### CLAIM OF PRIORITY

This application claims the benefit of priority under 35 U.S.C. § 119(e) of U.S. Provisional Patent Application Ser. No. 62/447,680, filed on Jan. 18, 2017, which is herein incorporated by reference in its entirety.

### TECHNICAL FIELD

This document relates generally to medical devices, and more particularly, but not by way of limitation, to systems, devices, and methods to regulate a melatonin level in a patient.

### BACKGROUND

Sleep disorders are widespread with approximately 6% of adults reporting symptoms of insomnia lasting for over a month and not associated with other disorders or events. The inability to sleep properly may lead to deficits in social and cognitive abilities and may also lead to the development of mental disorders. Current treatments for sleep disorders including insomnia may include lifestyle changes, medications, and external devices. Medications may be ineffective or may lead to dangerous dependencies (e.g., addiction). Lifestyle changes, such as reducing caffeine intake and exercising more frequently may not work for all patients. External devices may be inconvenient or uncomfortable to use, and moreover, may not work for all patients.

### SUMMARY

This document discusses, among other things, systems and methods to regulate a melatonin level in a patient. The ability to sleep is heavily regulated by activity levels of the pineal gland, which secretes melatonin into the body. Melatonin is a hormone used to signal the onset of night and, thus, a sleeping period. An oral medication of melatonin may be effective in treating a number of sleep disorders, including insomnia and delayed sleep phase syndrome. However, oral medication may undesirably reduce the amount of control a patient has over their sleep schedule, particularly when waking up from sleep.

An example, (e.g., “Example 1”) of subject matter (e.g., a system) may include an electrode assembly configured for use to electrically modulate a superior cervical ganglion or ganglia of a patient. The system may also include control circuitry configured to control electrical modulation of a patient's pineal gland and regulate melatonin levels of the patient by controlling a neuromodulation waveform to be delivered using the electrode assembly to electrically modulate the superior cervical ganglion or ganglia of the patient.

In Example 2, the subject matter of Example 1 may optionally include feedback circuitry configured to receive at least one feedback signal indicative of a patient melatonin level and provide the feedback signal to the control circuitry, wherein the control circuitry is further configured to adjust a value of at least one modulation parameter of the neuromodulation waveform to adjust the patient melatonin level in response to the received at least one feedback signal.

In Example 3, the subject matter of any one or more of Examples 1-2 may optionally include stimulation circuitry configured to deliver the neuromodulation waveform to the at least one electrode of the patient.

In Example 4, the subject matter of any one or more of Examples 2-3 may be optionally configured such that at least one feedback signal includes at least one of a light level, a pulse rate, a patient position, a time of day, or a patient posture.

In Example 5, the subject matter of any one or more of Examples 2-4 may be optionally configured such that the at least one feedback signal includes an indicator of approaching morning and wherein the control circuitry is further configured to adjust the at least one stimulation parameter to provide a decreased melatonin level in response to the indicator of approaching morning.

In Example 6, the subject matter of any one or more of Examples 2-5 may be optionally configured such that the at least one feedback signal includes an indicator of approaching evening and wherein the control circuitry is further configured to adjust the at least one stimulation parameter to provide an increased melatonin level in response to the indicator of approaching evening.

In Example 7, the subject matter of Example 1 may optionally be configured to adjust the value of at least one modulation parameter of the neuromodulation waveform to adjust a patient melatonin level according to scheduling information provided by a user.

In Example 8, the subject matter of any one or more of Examples 1-7 may be optionally configured such that at least one electrode is implantable in the patient.

In Example 9, the subject matter of any one or more of Examples 1-7 may be optionally configured such that at least one electrode is external to the patient.

An example, (e.g., “Example 10”) of subject matter (e.g., a method) may include electrically modulating a patient's pineal gland to control release of melatonin from the pineal gland and regulate melatonin levels of the patient, the electrically modulating the patient's pineal gland including delivering a neuromodulation waveform using at least one electrode to modulate a superior cervical ganglion or ganglia.

In Example 11, the subject matter of Example 10 may optionally include receiving at least one feedback signal indicative of a patient melatonin level and adjusting the value of at least one modulation parameter of the neuromodulation waveform to adjust the patient melatonin level in response to the received at least one feedback signal.

In Example 12, the subject matter of Example 11 may optionally be configured such that the at least one feedback signal includes at least one signal indicative of a light level.

In Example 13, the subject matter of any one or more of Examples 11-12 may optionally be configured such that the at least one feedback signal includes at least one signal indicative of a time of day.

In Example 14, the subject matter of any one or more of Examples 11-13 may optionally be configured such that the at least one feedback signal includes at least one signal indicative of a pulse rate, a patient position, or a patient posture.

In Example 15, the subject matter of any one or more of Examples 11-14 may optionally be configured such that the at least one feedback signal includes an indicator of approaching morning and the at least one stimulation parameter is adjusted to provide a decreased melatonin level in response to the indicator of approaching morning.

An example, (e.g., “Example 16”) of subject matter (e.g., a method) may include electrically modulating a patient's pineal gland to control release of melatonin from the pineal gland and regulate melatonin levels of the patient, the electrically modulating the patient's pineal gland including delivering a neuromodulation waveform using at least one electrode to modulate a superior cervical ganglion or ganglia.

In Example 17, the subject matter of Example 16 may optionally include receiving at least one feedback signal indicative of a patient melatonin level and adjusting the value of at least one modulation parameter of the neuromodulation waveform to adjust the patient melatonin level in response to the received at least one feedback signal.

In Example 18, the subject matter of Example 17 may optionally be configured such that the at least one feedback signal includes at least one signal indicative of a light level.

In Example 19, the subject matter of Example 17 may optionally be configured such that the at least one feedback signal includes at least one signal indicative of a time of day.

In Example 20, the subject matter of Example 17 may optionally be configured such that the at least one feedback signal includes at least one signal indicative of a pulse rate, a patient position, or a patient posture.

In Example 21, the subject matter of Example 17 may optionally be configured such that the at least one feedback signal includes an indicator of approaching morning and the at least one stimulation parameter is adjusted to provide a decreased melatonin level in response to the indicator of approaching morning.

In Example 22, the subject matter of Example 17 may optionally be configured such that the at least one feedback signal includes an indicator of approaching evening and the at least one stimulation parameter is adjusted to provide an increased melatonin level in response to the indicator of approaching evening.

In Example 23, the subject matter of Example 16 may optionally include receiving scheduling information indicative of a user provided schedule and adjusting a value of the at least one modulation parameter of the neuromodulation waveform to adjust a patient melatonin level according to the received scheduling information.

In Example 24, the subject matter of Example 16 may optionally be configured such that the electrically modulating the patient's pineal gland includes subcutaneously modulating the patient's pineal gland.

In Example 25, the subject matter of Example 16 may optionally be configured such that the electrically modulating the patient's pineal gland includes transcutaneously modulating the patient's pineal gland.

An example, (e.g., “Example 26”) of subject matter (e.g., a system) may include an electrode assembly configured for use to electrically modulate a superior cervical ganglion or ganglia of a patient. The system may also include control circuitry configured to control electrical modulation of a patient's pineal gland and regulate melatonin levels of the patient by controlling a neuromodulation waveform to be delivered using the electrode assembly to electrically modulate the superior cervical ganglion or ganglia of the patient.

In Example 27, the subject matter of claim 26 may optionally include feedback circuitry configured to receive at least one feedback signal indicative of a patient melatonin level and provide the feedback signal to the control circuitry, wherein the control circuitry is further configured to adjust a value of at least one modulation parameter of the neuromodulation waveform to adjust the patient melatonin level in response to the received at least one feedback signal.

In Example 28, the subject matter of claim 26 may optionally include stimulation circuitry configured to deliver the neuromodulation waveform to the at least one electrode of the patient.

In Example 29, the subject matter of claim 27 may optionally be configured such that the at least one feedback signal includes at least one of a light level, a pulse rate, a patient position, a time of day, or a patient posture.

In Example 30, the subject matter of claim 27 may optionally be configured such that the at least one feedback signal includes an indicator of approaching morning and wherein the control circuitry is further configured to adjust the at least one stimulation parameter to provide a decreased melatonin level in response to the indicator of approaching morning.

In Example 31, the subject matter of claim 27 may optionally be configured such that the at least one feedback signal includes an indicator of approaching evening and wherein the control circuitry is further configured to adjust the at least one stimulation parameter to provide an increased melatonin level in response to the indicator of approaching evening.

In Example 32, the subject matter of claim 27 may optionally be configured to adjust the value of at least one modulation parameter of the neuromodulation waveform to adjust a patient melatonin level according to scheduling information provided by the user.

In Example 33, the subject matter of claim 27 may optionally be configured such that the at least one electrode is implantable in the patient.

In Example 34, the subject matter of claim 27 may optionally be configured such that the at least one electrode is external to the patient.

An example, (e.g., “Example 35”) of subject matter (e.g., a system) may include a non-transitory machine-readable medium including instructions, which when executed by a machine, cause the machine to control electrical modulation of a patient's pineal gland and regulate melatonin levels of the patient by controlling a neuromodulation waveform to be delivered using an electrode assembly to modulate the patient's pineal gland by electrically modulating a superior cervical ganglion or ganglia of the patient.

An example (e.g., “Example 36”) of subject matter (e.g., a system or apparatus) may optionally combine any portion or combination of any portion of any one or more of Examples 1-35 to include “means for” performing any portion of any one or more of the functions or methods of Examples 1-35, or a “machine-readable medium” (e.g., massed, non-transitory, etc.) including instructions that, when performed by a machine, cause the machine to perform any portion of any one or more of the functions or methods of Examples 1-35.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates an example of a patient's pineal gland and related neural anatomy.

FIG. 1B illustrates an example of a patient's cervical sympathetic trunk and ganglia, including the patient's superior cervical ganglion and carotid artery.

FIG. 1C illustrates an example of a root of a patient's neck.

FIG. 1D illustrates an example of a neurostimulation system.

FIG. 2 illustrates an example of a stimulation device.

FIG. 3 illustrates an example of a programming device.

FIG. 4 illustrates an example of an implantable neurostimulation system.

FIG. 5A illustrates an example method for regulating a melatonin level in a patient.

FIG. 5B illustrates an example method for regulating a melatonin level in a patient.

FIG. 6A illustrates an example of a measured melatonin level in a healthy patient.

FIG. 6B illustrates a table including a time of day and a target melatonin level.

FIG. 7A illustrates an example of an external patch stimulator.

FIG. 7B illustrates an example of an external collar stimulator.

FIG. 8A illustrates an example of an implantable electrode.

FIG. 8B illustrates an example of an implantable electrode.

FIG. 8C illustrates an example of an implantable electrode.

FIG. 8D illustrates an example of an implantable lead.

FIG. 8E illustrates an example of an implantable lead.

FIG. 8F illustrates an example of an implantable lead.

FIG. 8G illustrates an example of an implantable lead.

FIG. 8H illustrates an example of an implantable lead.

FIG. 8I illustrates an example of an implantable lead.

FIG. 9 illustrates a block diagram of an example machine upon which any one or more of the techniques (e.g., methodologies) discussed herein may be performed.

### DETAILED DESCRIPTION

A system and method are provided for regulating the production of melatonin in a patient, to provide treatment of a sleep disorder, such as insomnia, delayed sleep phase disorder, advanced sleep phase disorder, jet lag, shift work disorder, and narcolepsy. The superior cervical ganglion innervates the pineal gland and may provide a first target for neuromodulation to relieve sleep disorders. Preganglionic neurons located in the lateral horn of the spinal cord at segments C1-C4 may provide a second target for neuromodulation to relieve sleep disorders. Stimulation of the superior cervical ganglion or the preganglionic neurons in the lateral horn, may lead to increased melatonin levels. The inventors have recognized among other things, that providing neurostimulation to neurons and their associated nerves in proximity to the pineal gland may provide an improved system for regulating a melatonin level in a patient.

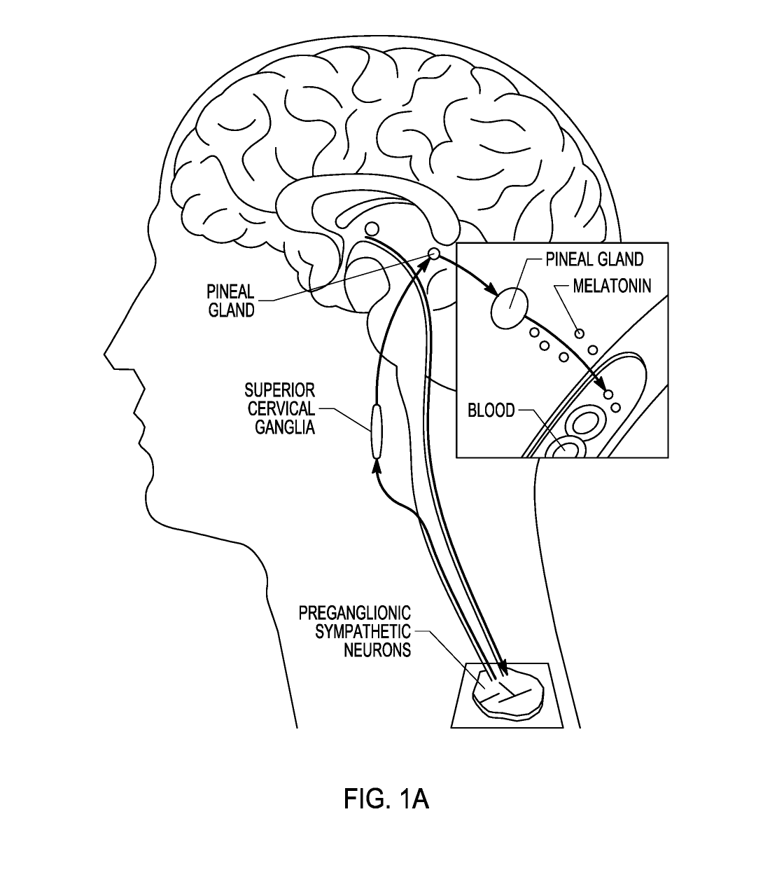

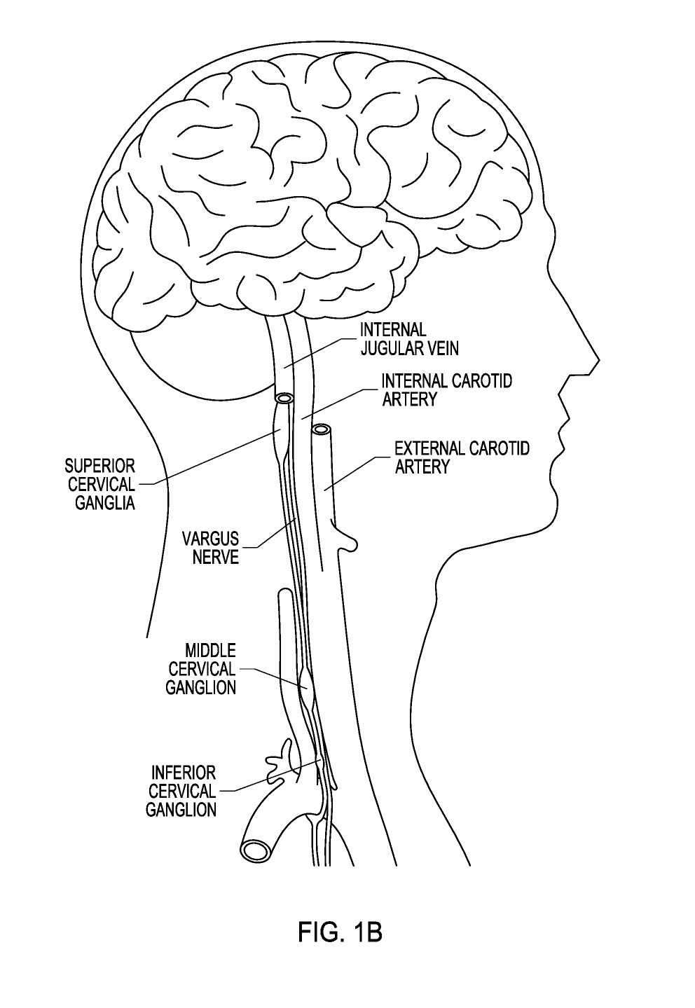

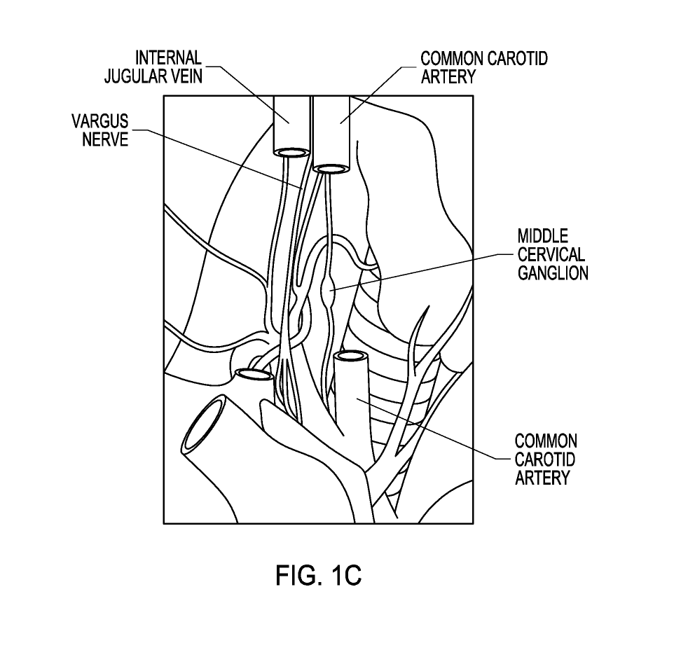

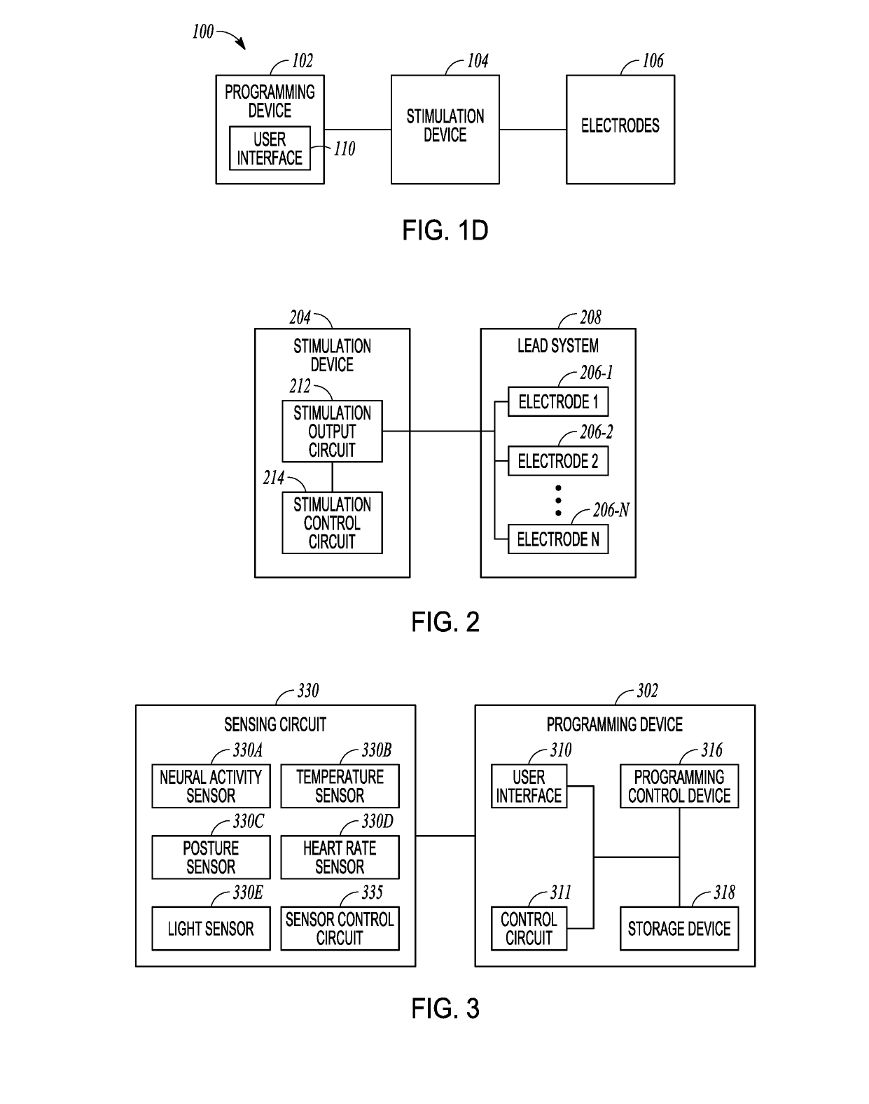

FIG. 1A illustrates an example of a patient's pineal gland and related neural anatomy. The patient's pineal gland may produce melatonin and provide the melatonin to the patient's blood stream, such as to regulate a sleep cycle of the patient. In the example a first neural pathway may include a connection from the superior cervical ganglion to the patient's pineal gland. In the example, a second neural pathway may include a connection from preganglionic sympathetic neurons (e.g., preganglionic sympathetic neurons in proximity to spinal cord segments C1-C4) to the patient's pineal gland. Delivery of stimulation to either the superior cervical ganglion or the preganglionic sympathetic neurons can provide stimulation to the patient's pineal gland. In an example, the pineal gland may be directly stimulated, such as by using a focused ultrasound system to directly target the pineal gland. FIG. 1B illustrates an example of a patient's cervical sympathetic trunk and ganglia, including the patient's superior cervical ganglion and carotid artery. FIG. 1C illustrates an example of a root of a patient's neck.

FIG. 1D illustrates an example of a neurostimulation system 100. The neurostimulation system 100 may include electrodes 106, a stimulation device 104, and a programming device 102. The electrodes 106 may be configured to be placed on or near one or more neural targets in a patient. The stimulation device 104 may be configured to be electrically connected to the electrodes 106 and deliver neurostimulation energy, such as in the form of an electrical waveform, to the one or more neural targets though the electrodes 106. The delivery of the neurostimulation may be controlled using a plurality of stimulation parameters, such as stimulation parameters specifying a waveform shape such as, but not limited to, a pattern of electrical pulses and a selection of electrodes through which each of the electrical pulses may be delivered. The stimulation parameters may include a pulse width, a duty cycle, an amplitude, or a frequency. At least some parameters of the plurality of stimulation parameters may be programmable by a user, such as a physician or other caregiver who treats the patient using the neurostimulation system 100. The programming device 102 may provide the user with accessibility to the user-programmable parameters. The programming device 102 may be configured to be communicatively coupled to the stimulation device 104 via a wired or wireless link. The programming device 102 may receive a signal from the patient and based on the received signal, the programming device 102 may automatically adjust the stimulation parameters, such as to provide a controlled release of melatonin from the patient's pineal gland. In an example, the received signal may include information indicative of a melatonin level within the patient.

In an example, the programming device 102 may include a user interface that allows the user to set and/or adjust values of the user-programmable parameters by creating and/or editing graphical representations of various waveforms. In an example, the user-programmable parameters may include a pulse width, a duty cycle, an amplitude, or a frequency. Such waveforms may include different waveform shapes. The waveform shapes may include regular shapes (e.g. square, sinusoidal, triangular, saw tooth, and the like) or irregular shapes. Such waveforms may include, for example, a pattern of neurostimulation pulses to be delivered to the patient. Examples of such neurostimulation pulses may include pulses, bursts each including a group of the pulses, trains each including a group of the bursts, and sequences each including a group of the pulses, bursts, and trains, as further discussed below. In the illustrated embodiment, the user interface may include a user interface 110. In various embodiments, the user interface 110 may include a graphical user interface (GUI) or any other type of user interface accommodating various functions including waveform composition as discussed in this document.

FIG. 2 illustrates an example of a stimulation device 204 and a lead system 208, such as may be implemented in the neurostimulation system 100. The stimulation device 204 may represent an example of the stimulation device 104 and may include a stimulation output circuit 212 and a stimulation control circuit 214. The stimulation output circuit 212 may produce and deliver a neurostimulation waveform. Such waveforms may include different waveform shapes. The waveform shapes may include regular shapes (e.g. square, sinusoidal, triangular, saw tooth, and the like) or irregular shapes. The stimulation control circuit 214 may control the delivery of the neurostimulation waveform using the plurality of stimulation parameters, which specifies a pattern of the neurostimulation waveform. In an example, the stimulation parameters may include a pulse width, a duty cycle, an amplitude, or a frequency. The lead system 208 may include one or more leads each configured to be electrically connected to the stimulation device 204 and a plurality of electrodes 206 distributed in the one or more leads. The plurality of electrodes 206 may include electrode 206-1, electrode 206-2, . . . electrode 206-N, each a single electrically conductive contact providing for an electrical interface between the stimulation output circuit 212 and the tissue of the patient, where N≥2. The neurostimulation waveform may be delivered from stimulation output circuit 212 through a set of electrodes selected from electrodes 206. In an example, the number of leads and the number of electrodes on each lead depend on, for example, the distribution of target(s) of the neurostimulation and the need for controlling the distribution of electric field at each target. In an example, the lead system 208 may include 2 leads each having 8 electrodes.

FIG. 3 illustrates an example of a programming device 302, such as may be implemented in the neurostimulation system 100. The programming device 302 may represent an embodiment of the programming device 102 and may include a storage device 318, a programming control circuit 316, a control circuit 311 and a user interface 310. The storage device 318 may store a plurality of neurostimulation waveforms. The programming control circuit 316 may generate a plurality of stimulation parameters that control the delivery of the neurostimulation waveform according to the pattern of the neurostimulation waveform. The neurostimulation waveform may be delivered to at least one electrode, such as to stimulate a patient's pineal gland. In an example the neurostimulation waveform may be delivered to the patient's superior cervical ganglion, such as to provide stimulation to the patient's pineal gland. In an example, the neurostimulation waveform may be delivered to the patient's preganglionic neurons located in the lateral horn at segments C1-C4, such as to provide stimulation of the patient's pineal gland. In an example, the neurostimulation waveform may be delivered directly to the patient's pineal gland, such as by using a focused ultrasound system to directly target the pineal gland. In an example, the programming device 302 may be a mobile device, such as a mobile phone.

The control circuit 311 may receive a signal indicative of a patient melatonin level and may adjust the value of at least one of the plurality of stimulation parameters based on the received signal. In an example, a sensing circuit 330 may provide the signal indicative of a patient melatonin level to the control circuit 311. The sensing circuit 330 may include a neural activity sensor 330a, a temperature sensor 330b, a posture sensor 330c, a heart rate sensor 330d, a light sensor 330e, a melatonin sensor 330f, and a sensor control circuit 335. The neural activity sensor 330a may include an external or subdural electroencephalogram (EEG) sensor. The neural sensor 330a may sense an electrocorticography (ECoG) signal or a local field potential (LFP) signal, or an evoked compound action potential (eCAP). The temperature sensor 330b may sense a temperature of a patient's skin. The patient's skin temperature may provide an indication of patient sleep. The heart rate sensor 330d may include an accelerometer, a photoplethysmography sensor, an electrocardiogram (ECG) sensor, an electrical bio-impedance sensor, an impedance cardiography sensor, or a pressure sensor. The posture sensor 330c may include an accelerometer or a gyroscope. The melatonin sensor 330f may sense a level of melatonin in the patient's blood. In an example, the melatonin sensor 330f may include a biosensor implanted within the patient. In an example, the light sensor 330e may be included in an external device (e.g., a mobile phone) such as the programming device 302. The sensing circuit 330 may include an electrodermal activity (EDA) sensor, such as to determine an electrical characteristic of a patient's skin. The received signal may include a measure of brain activity, neural activity, a respiratory rate, a pulse rate, an automatic tone, a patient position, a patient posture, a time of day, a patient skin temperature, or a light level. The control circuit 311 may determine an indicator of patient sleep based on the received signal. For example, the control circuit 311 may determine a sleep state of the patient based on at least one of a brain activity, a respiratory rate, a pulse rate, an automatic tone, a patient position, a time of day, a patient skin temperature, or a light level. In an example, the control circuity 311 may adjust at least one stimulation parameter to provide a decreased melatonin level in response to receiving an indicator of approaching morning from the sensing circuit 330. In an example, the control circuity 311 may adjust at least one stimulation parameter to provide an increased melatonin level in response to receiving an indicator of approaching evening from the sensing circuit 330. In an example, the programming device 302 may include a clock, such as to provide a local time, such as to allow the control circuit to make adjustments to a patient's melatonin level based on a local light level. In an example, the sensor control circuit 335 may receive inputs from at least one of the sensors 330a-330f. The sensor control circuit 335 may then determine at least one stimulation parameter based on the received inputs from the at least one sensor. The sensor control circuit 335 may compute a statistical quantity, such as a heart rate variability based on the received inputs. The sensor control circuit 335 may then determine at least one stimulation parameter based on the computed statistical quantity and the received inputs from the at least one sensor. In an example, the sensor control circuit 335 may determine at least one stimulation parameter using at least one of heart rate variability, posture, time, patient input, heart rate, respiration rate, sympathetic activity such as can be determined by an EDA sensor, a sleep state, and light levels.

In an example, the user interface 310 may include, but is not limited to, a touchscreen. In an example, the user interface 310 may include any type of presentation device, such as interactive or non-interactive screens, and any type of user input devices that allow the user to edit the waveforms or building blocks and schedule the programs, such as touchscreen, keyboard, keypad, touchpad, trackball, joystick, and mouse. In an example, the user interface 310 may include a calendar, such as to allow a user to program times for a sleeping schedule. In an example, the circuits of neurostimulation system 100, including its various embodiments discussed in this document, may be implemented using a combination of hardware and software. For example, the circuit of the user interface 110, the stimulation control circuit 214, and the programming control circuit 316, including their various embodiments discussed in this document, may be implemented using an application-specific circuit constructed to perform one or more particular functions or a general-purpose circuit programmed to perform such function(s). Such a general-purpose circuit may include, but is not limited to, a microprocessor or a portion thereof, a microcontroller or portions thereof, and a programmable logic circuit or a portion thereof.

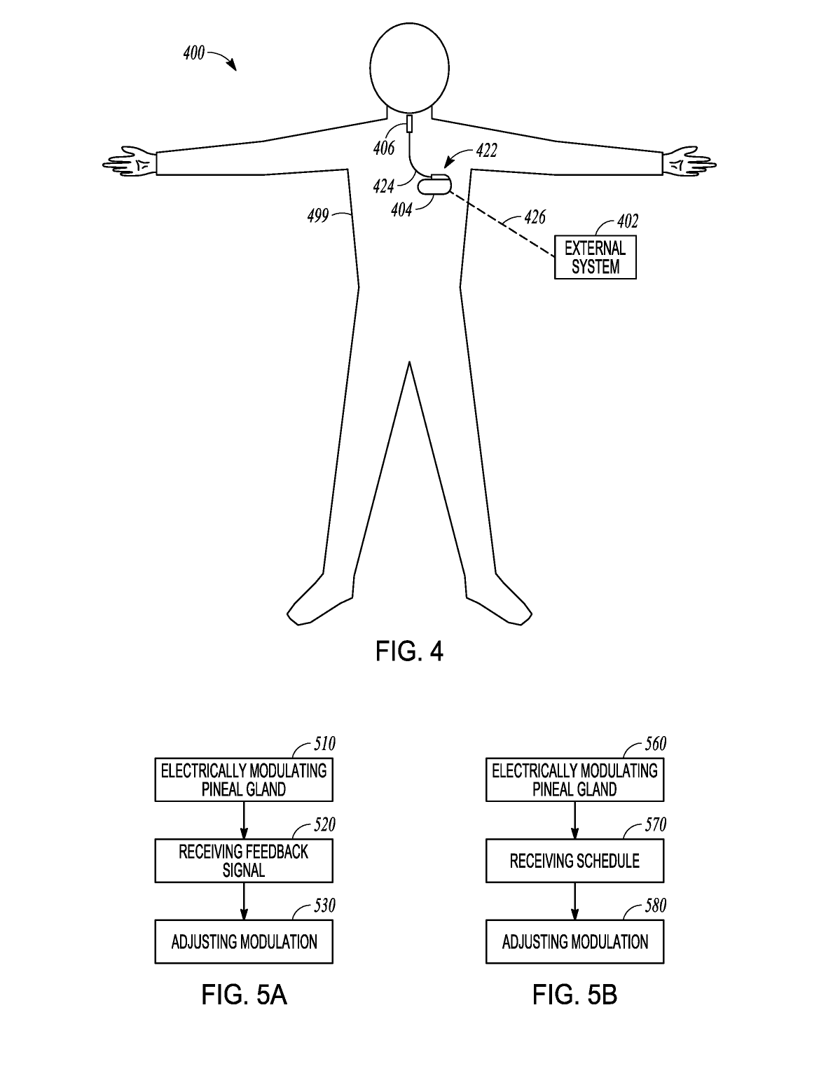

FIG. 4 illustrates, by way of example and not limitation, an implantable neurostimulation system 400 and portions of an environment in which system 400 may be used. The system 400 may include an implantable system 422, an external system 402, and a telemetry link 426 providing for wireless communication between implantable system 422 and external system 402. The implantable system 422 is illustrated in FIG. 4 as being implanted in the patient's body 499.

The implantable system 422 may include an implantable stimulator (also referred to as an implantable pulse generator, or IPG) 404, a lead system 424, and electrodes 406, which may represent an embodiment of stimulation device 204, lead system 208, and electrodes 206, respectively. The external system 402 may represent an embodiment of programming device 302. In an example, the external system 402 includes one or more external (non-implantable) devices each allowing the user and/or the patient to communicate with implantable system 422. In an example, the external system 402 may include a programming device intended for the user to initialize and adjust settings for the implantable stimulator 404 and a remote control device intended for use by the patient. For example, the remote control device may allow the patient to turn the implantable stimulator 404 on and off and/or adjust certain patient-programmable parameters of the plurality of stimulation parameters.

The sizes and shapes of the elements of the implantable system 422 and their location in the body 499 are illustrated by way of example and not by way of restriction. In various examples, the present subject matter may be applied in programming any type of stimulation device that uses electrical waveforms or electrical pulses as stimuli, regardless of stimulation targets in the patient's body and whether the stimulation device is implantable.

FIG. 5A illustrates an example of a method for regulating a melatonin level in a patient. A neurostimulation system, such as neurostimulation system 100 may provide a neuromodulation waveform to at least one electrode to modulate a superior cervical ganglion or preganglionic neurons located in the lateral horn at segments C1-C4 or the associated nerves of either location, such as to control release of melatonin from the patient's pineal gland (step 510). In an example, the neurostimulation system 100 may provide a neuromodulation waveform to a focused ultrasound system, such as to directly target the patient's pineal gland. A control circuit, such as control circuit 311 may receive a feedback signal indicative of a patient melatonin level (step 520). The feedback signal may include a signal indicative of a light level, a signal indicative of a time of day, a signal indicative of a pulse rate, a signal indicative of a patient posture, a signal indicative of a patient position, or a signal indicative of a patient temperature. The control circuit may then adjust at least one stimulation parameter (e.g., an amplitude, a frequency, or a duty cycle) of the neuromodulation waveform, such as to adjust the patient melatonin level (step 530). A patient melatonin level can be determined, such as by the melatonin sensor 330f.

In an example, the feedback signal may include a signal that indicates if a patient is in a standing position. A posture sensor, such as the posture sensor 330c may provide the signal indicative of a patient in a standing position. If the feedback signal indicates that the patient is in a standing position, the control circuit 311 may adjust a stimulation parameter such as to provide a relatively low patient melatonin level, such as to prevent the patient from falling asleep while in the standing position.

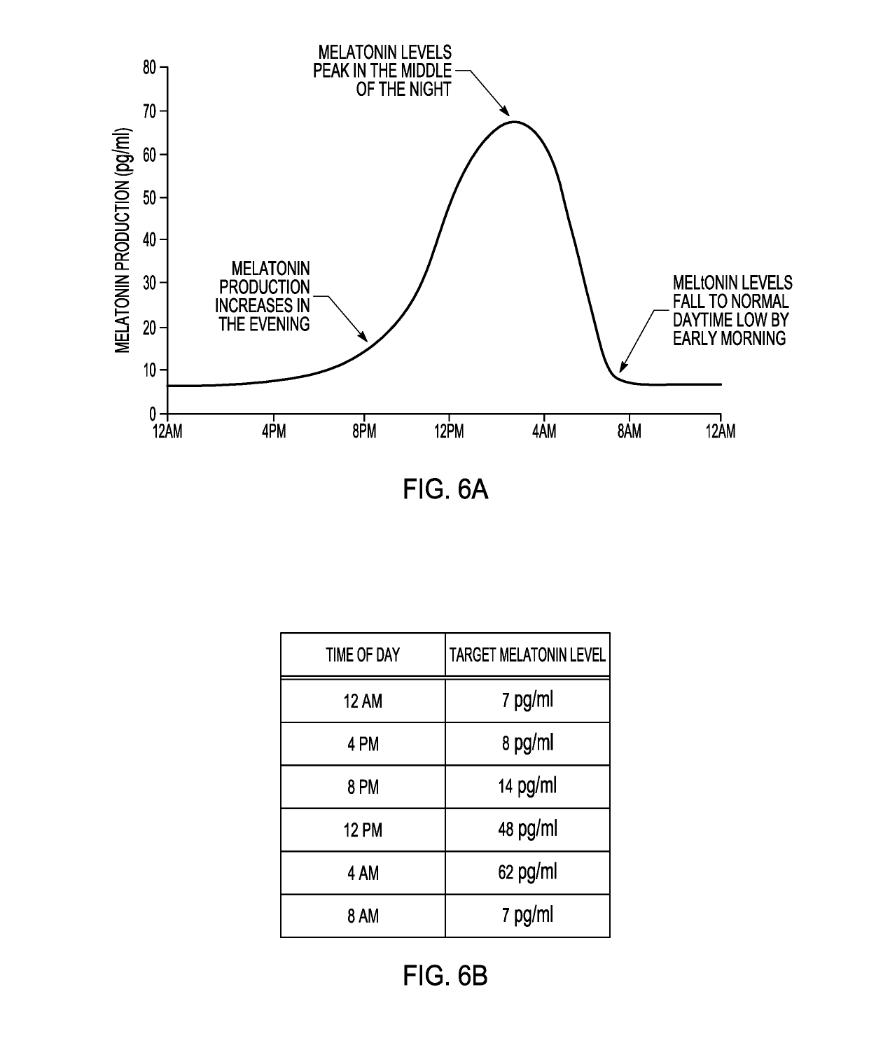

FIG. 5B illustrates an example of a method for regulating a melatonin level in a patient. A neurostimulation system, such as neurostimulation system 100 may provide a neuromodulation waveform to at least one electrode to modulate a superior cervical ganglion or preganglionic neurons located in the lateral horn at segments C1-C4 or the associated nerves of either location, such as to control release of melatonin from the patient's pineal gland (step 560). In an example, the neurostimulation system 100 may provide a neuromodulation waveform to a focused ultrasound system, such as to directly target the patient's pineal gland. A control circuit, such as control circuit 311 may receive a predetermined schedule indicative of a target patient melatonin level (step 570). The predetermined schedule may include a table including a time of day and a target melatonin level as shown in FIG. 6B. The control circuit may then adjust at least one stimulation parameter of the neuromodulation waveform, such as to adjust the patient melatonin level towards the target patient melatonin level (step 580). A patient melatonin level can be determined, such as by the melatonin sensor 330f.

FIG. 6B illustrates an example of a predetermined schedule indicative of a patient target melatonin level. The predetermined schedule may include a time of day and a patient target melatonin level. The time of day and the patient target melatonin level may be based on a measured melatonin level in a healthy patient as shown in FIG. 6A. In an example, the predetermined schedule may include a patient target melatonin level at one minute intervals, five minute intervals, ten minute intervals, thirty minute intervals, one hour intervals, two hour intervals, or four hour intervals. The patient target melatonin level may vary significantly among patients.

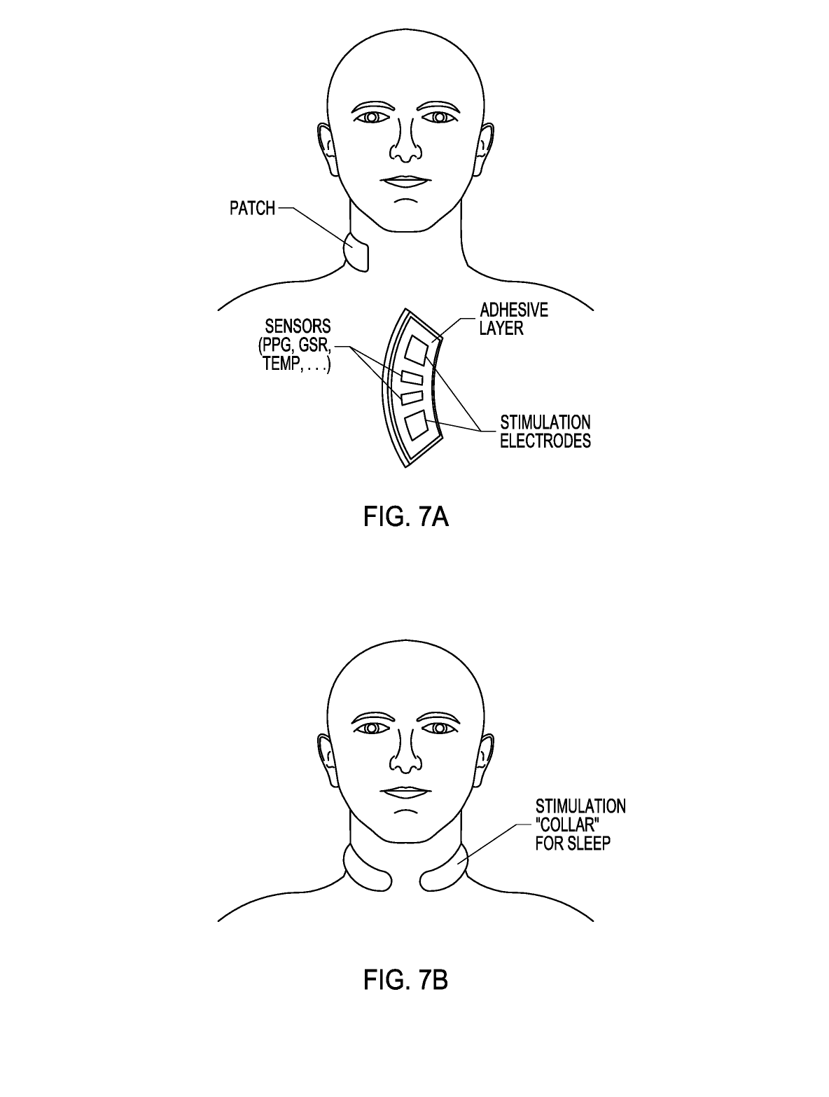

FIG. 7A illustrates an example of an external patch stimulator. The external patch stimulator may include an adhesive patch and may be removably adhered to a patient's neck. In an example, the external patch stimulator may be adhered to the patient's neck beneath the patient's ear. The external patch stimulator may include external stimulation electrodes, a programming device, such as the programming device 302, and a sensing circuit, such as the sensing circuit 330. During operation, the external patch stimulator may regulate a melatonin level of a patient as describe in FIGS. 5A and 5B. In an example, the external patch stimulator may remotely control at least one implanted electrode.

FIG. 7B illustrates an example of an external collar stimulator. The external collar stimulator may be worn comfortably around a patient's neck. In an example, the external collar stimulator may be located at the back of a patient's neck. The external collar stimulator may include external stimulation electrodes, a programming device, such as the programming device 302, and a sensing circuit, such as the sensing circuit 330. During operation, the external collar stimulator may regulate a melatonin level of a patient as describe in FIGS. 5A and 5B. In an example, the external patch stimulator may remotely control at least one implanted electrode.

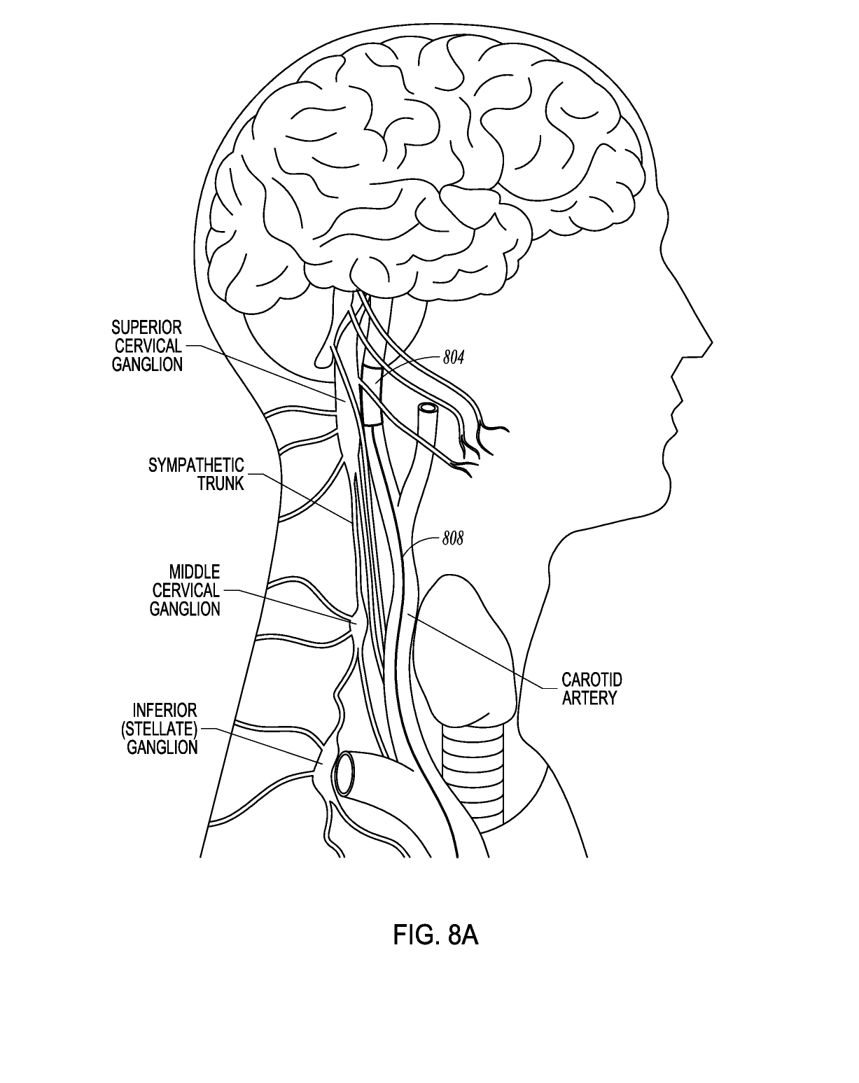

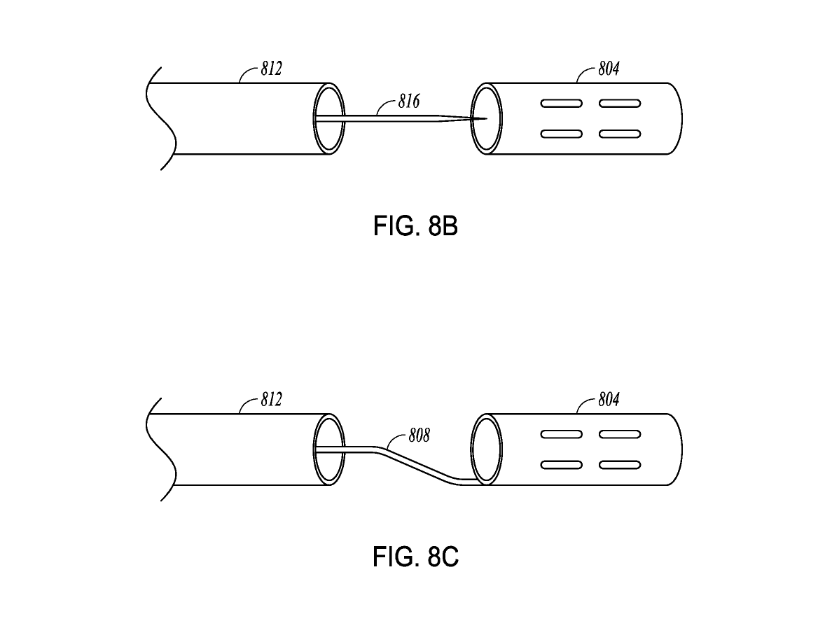

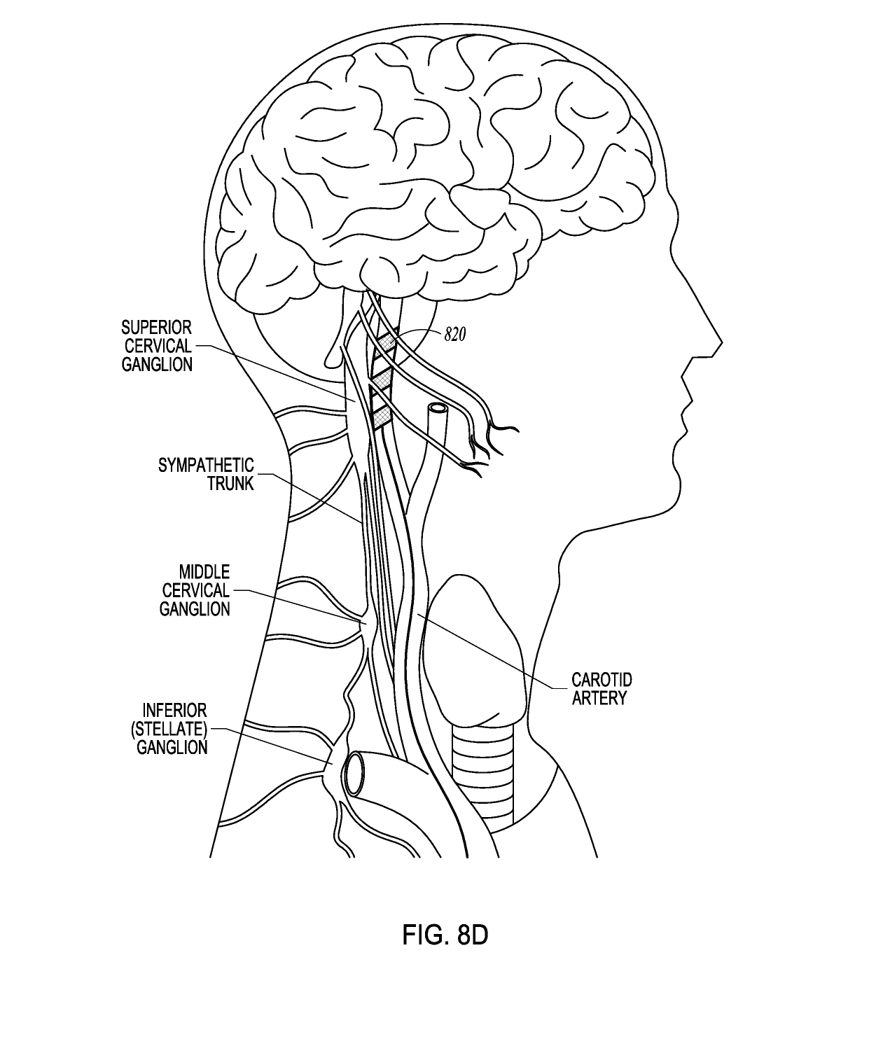

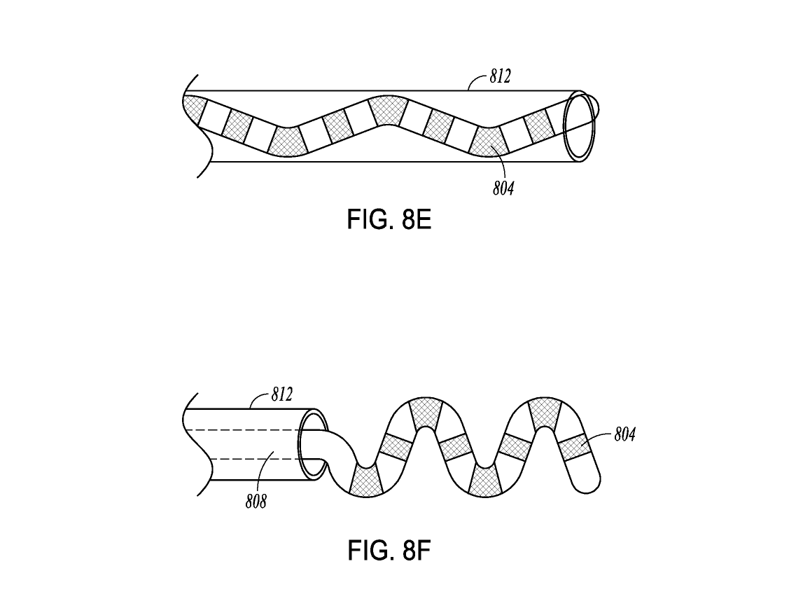

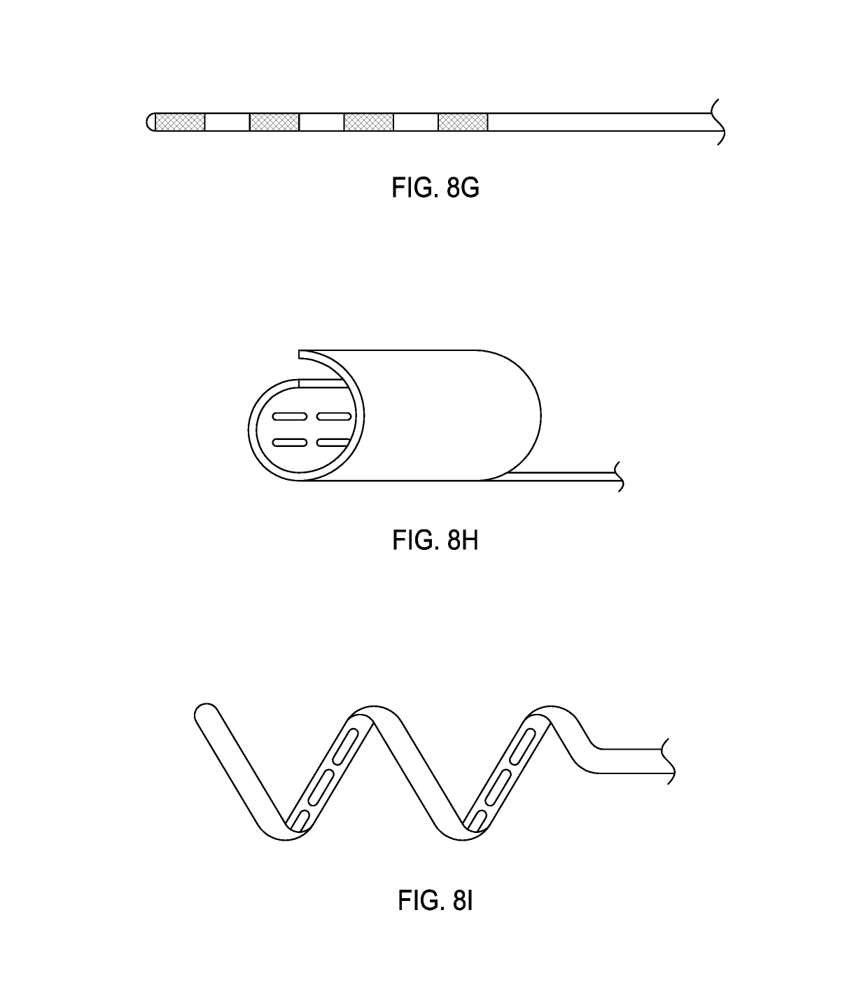

FIG. 8A illustrates an example of an electrode 804, such as that may be placed proximal to a patient's superior cervical ganglion. The electrode 804 may be connected to an implantable pulse generator, such as IPG 404, by a lead, such as a lead 808. In an example, the electrode 804 may be placed proximal to a patient's superior cervical ganglion by channeling the lead 808 along a patient's internal carotid sheath. In an example, the electrode 804 and the lead 808 may be placed proximal to the patient's superior cervical ganglion extravascularly using a percutaneous approach. In an example, the electrode 804 and the lead 808 may be placed proximal to a patient's superior cervical ganglion intravascularly using a guide catheter, such as guide catheter 812.

FIG. 8B illustrates an example where the electrode 804 may be placed proximal to the patient's superior cervical ganglion by (i) delivering a guide catheter 812, a balloon catheter 816, and a lead 808 to a target in a vessel; (ii) expanding the balloon catheter 812 to expand the electrode 804 to interface with the vessel; (iii) deflating and removing the balloon catheter 816 and removing the guide catheter 812; and (iv) leaving the lead 808 at the target and implant an implantable pulse generator in the patient, for example in a chest pocket. FIG. 8C illustrates an example of an implanted electrode 804 where the balloon catheter 816 has been deflated and removed. FIG. 8D illustrates an example where an implantable lead 820 may be proximal to a patient's superior cervical ganglion.

FIG. 8E illustrates an example where the lead 820 may include a self-expanding lead. The self-expanding lead 820 may have a helical shape. The self-expanding lead 820 may be delivered with a guide catheter 812. The guide catheter 812 may be delivered to a target in a vessel. The self-expanding lead 820 may then be translated out of the guide catheter 812. After exiting the guide catheter 812, the self-expanding lead 820 may expand to interface with an inner diameter of the vessel as shown in FIG. 8F. The guide catheter 812 may then be removed from the vessel, leaving the self-expanding lead 820 at the target. An implantable pulse generator may be implanted subcutaneously in the patient and connected to the self-expanding lead 820.

FIG. 8G illustrates an example where the lead 820 may include a substantially straight lead. The substantially straight lead 820 may be delivered through a patient's internal carotid sheath to a location proximal to a patient's superior cervical ganglion. The substantially straight lead 820 may be tunneled to a subcutaneous pocket for connection to an implantable pulse generator.

FIG. 8H illustrates an example where the lead 820 may include a cuff. The cuff may have a slotted cylindrical shape that may be configured to wrap around a vessel or nerve of a patient. The cuff may be delivered surgically or percutaneously. The lead 820 may be tunneled to a chest pocket for connection to an implantable pulse generator. In an example, the lead 820 may include a helical cuff that may be configured to wrap around a vessel or nerve of a patient as shown in FIG. 8I. In an example, a lead connected to the cuff may be delivered surgically or percutaneously. The lead may be tunneled to a subcutaneous pocket for connection to an implantable pulse generator.

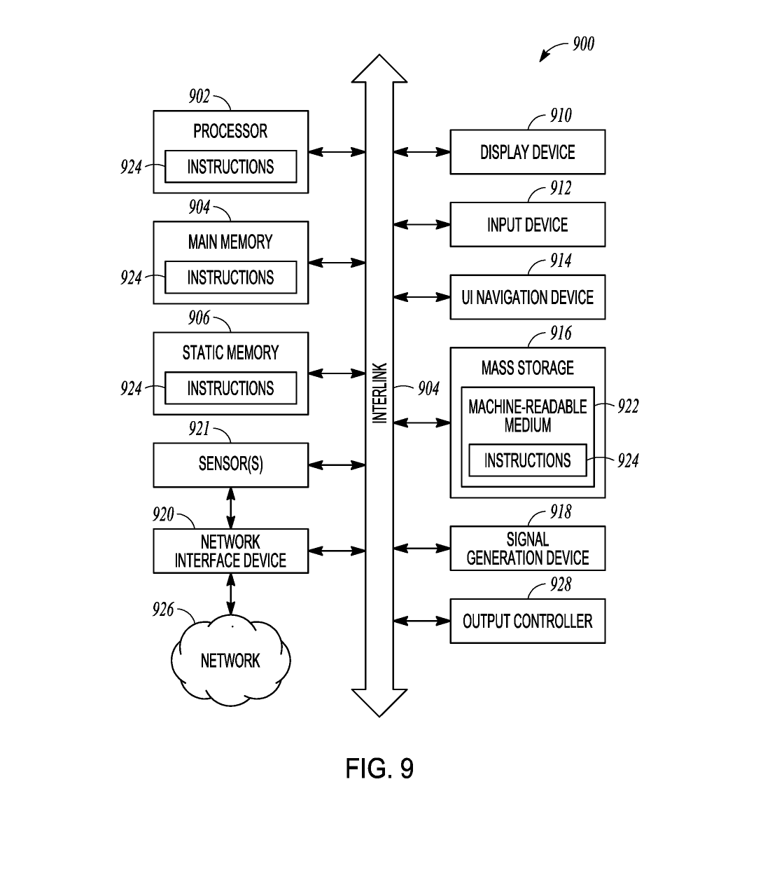

FIG. 9 illustrates generally a block diagram of an example machine 900 upon which any one or more of the techniques (e.g., methodologies) discussed herein may perform. Portions of this description may apply to the computing framework of various portions of the LCP device, the IMD, or the external programmer.

In alternative embodiments, the machine 900 may operate as a standalone device or may be connected (e.g., networked) to other machines. In a networked deployment, the machine 900 may operate in the capacity of a server machine, a client machine, or both in server-client network environments. In an example, the machine 900 may act as a peer machine in peer-to-peer (P2P) (or other distributed) network environment. The machine 900 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 900 may include a hardware processor 902 (e.g., a central processing unit (CPU), a graphics processing unit (GPU), a hardware processor core, or any combination thereof), a main memory 904 and a static memory 906, some or all of which may communicate with each other via an interlink (e.g., bus) 908. The machine 900 may further include a display unit 910 (e.g., a raster display, vector display, holographic display, etc.), an alphanumeric input device 912 (e.g., a keyboard), and a user interface (UI) navigation device 914 (e.g., a mouse). In an example, the display unit 910, input device 912 and UI navigation device 914 may be a touch screen display. The machine 900 may additionally include a storage device (e.g., drive unit) 916, a signal generation device 918 (e.g., a speaker), a network interface device 920, and one or more sensors 921, such as a global positioning system (GPS) sensor, compass, accelerometer, or other sensor. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 916 may include a machine readable medium 922 on which is stored one or more sets of data structures or instructions 924 (e.g., software) embodying or utilized by any one or more of the techniques or functions described herein. The instructions 924 may also reside, completely or at least partially, within the main memory 904, within static memory 906, or within the hardware processor 902 during execution thereof by the machine 900. In an example, one or any combination of the hardware processor 902, the main memory 904, the static memory 906, or the storage device 916 may constitute machine readable media.

While the machine readable medium 922 is illustrated as a single medium, the term “machine readable medium” may include a single medium or multiple media (e.g., a centralized or distributed database, and/or associated caches and servers) configured to store the one or more instructions 924.

The term “machine readable medium” may include any medium that is capable of storing, encoding, or carrying instructions for execution by the machine 900 and that cause the machine 900 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 924 may further be transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a low-power wide area network (LPWAN) a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 900, and includes digital or analog communications signals or other intangible medium to facilitate communication of such software.

Various embodiments are illustrated in the figures above. One or more features from one or more of these embodiments may be combined to form other embodiments.

Method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method comprising:
electrically modulating a patient's pineal gland to control release of melatonin from the pineal gland and regulate melatonin levels of the patient, the electrically modulating the patient's pineal gland including delivering a neuromodulation waveform using at least one electrode to modulate a superior cervical ganglion or ganglia.

2. The method of claim 1, further comprising:
receiving at least one feedback signal indicative of a patient melatonin level; and
adjusting the value of at least one modulation parameter of the neuromodulation waveform to adjust the patient melatonin level in response to the received at least one feedback signal.

3. The method of claim 2, wherein the at least one feedback signal includes at least one signal indicative of a light level.

4. The method of claim 2, wherein the at least one feedback signal includes at least one signal indicative of a time of day.

5. The method of claim 2, wherein the at least one feedback signal includes at least one signal indicative of a pulse rate, a patient position, or a patient posture.

6. The method of claim 2, wherein the at least one feedback signal includes an indicator of approaching morning and the at least one stimulation parameter is adjusted to provide a decreased melatonin level in response to the indicator of approaching morning.

7. The method of claim 2, wherein the at least one feedback signal includes an indicator of approaching evening and the at least one stimulation parameter is adjusted to provide an increased melatonin level in response to the indicator of approaching evening.

8. The method of claim 1, further comprising:
receiving scheduling information indicative of a user provided schedule; and
adjusting a value of the at least one modulation parameter of the neuromodulation waveform to adjust a patient melatonin level according to the received scheduling information.

9. The method of claim 1, wherein the electrically modulating the patient's pineal gland includes subcutaneously modulating the patient's pineal gland.

10. The method of claim 1, wherein the electrically modulating the patient's pineal gland includes transcutaneously modulating the patient's pineal gland.

11. A system comprising:
an electrode assembly configured for use to electrically modulate a superior cervical ganglion or ganglia of a patient; and
control circuitry configured to control electrical modulation of a patient's pineal gland and regulate melatonin levels of the patient by controlling a neuromodulation waveform to be delivered using the electrode assembly to electrically modulate the superior cervical ganglion or ganglia of the patient.

12. The system of claim 11, further comprising feedback circuitry configured to receive at least one feedback signal indicative of a patient melatonin level and provide the feedback signal to the control circuitry, wherein the control circuitry is further configured to adjust a value of at least one modulation parameter of the neuromodulation waveform to adjust the patient melatonin level in response to the received at least one feedback signal.

13. The system of claim 11, further comprising stimulation circuitry configured to deliver the neuromodulation waveform to the at least one electrode of the patient.

14. The system of claim 12, wherein the at least one feedback signal includes at least one of a light level, a pulse rate, a patient position, a time of day, or a patient posture.

15. The system of claim 12, wherein the at least one feedback signal includes an indicator of approaching morning and wherein the control circuitry is further configured to adjust the at least one stimulation parameter to provide a decreased melatonin level in response to the indicator of approaching morning.

16. The system of claim 12, wherein the at least one feedback signal includes an indicator of approaching evening and wherein the control circuitry is further configured to adjust the at least one stimulation parameter to provide an increased melatonin level in response to the indicator of approaching evening.

17. The system of claim 12, wherein the control circuitry is further configured to adjust the value of at least one modulation parameter of the neuromodulation waveform to adjust a patient melatonin level according to scheduling information provided by a user.

18. The system of claim 12, wherein the at least one electrode is implantable in the patient.

19. The system of claim 12, wherein the at least one electrode is external to the patient.

20. A non-transitory machine-readable medium including instructions, which when executed by a machine, cause the machine to control electrical modulation of a patient's pineal gland and regulate melatonin levels of the patient by controlling a neuromodulation waveform to be delivered using an electrode assembly to modulate the patient's pineal gland by electrically modulating a superior cervical ganglion or ganglia of the patient.
